(19) United States
(12) Patent Application Publication
(10) Pub. No.: US 2008/0019923 A1
(43) Pub. Date: Jan. 24, 2008

(54) GAS BUBBLE-GENERATING AGENT

(76) Inventors: Kenichi KAWABATA, Kodaira (JP); Koji Hirata, Kasukabe (JP); Kazuaki Sasaki, Kawasaki (JP)

Correspondence Address: MATTINGLY, STANGER, MALUR & BRUNDIDGE, P.C., 1800 DIAGONAL ROAD, SUITE 370, ALEXANDRIA, VA, 22314, US

(21) Appl. No.: 11/778,255

(22) Filed: Jul. 16, 2007

(30) Foreign Application Priority Data
Jul. 18, 2006 (JP) 2006-195666

(51) Int. Cl. A61B 8/00

(52) U.S. Cl. 424/9.52

(57) ABSTRACT

This invention provides a gas bubble-generating agent that can be used as a contrast medium or a blocking agent in vivo. Such gas bubble-generating agent is produced by a method for producing a gas bubble-generating agent comprising the following steps of: (a) preparing a mixed solution of an amphiphilic substance, an amphiphilic substance comprising a water-soluble polymer chemically bound thereto, a hardly water-soluble substance having a boiling point of lower than 60° C. at atmospheric pressure, and a physiologically acceptable isotonic solution; (b) pressurizing the mixed solution; and (c) centrifuging the mixed solution after the step of pressurization, wherein a molar concentration of the amphiphilic substance in the mixed solution prepared in step (a) is 10 times or more higher than that of the amphiphilic substance comprising a water-soluble polymer chemically bound thereto.

### CLAIM OF PRIORITY

The present application claims priority from Japanese application JP 2006-195666 filed on Jul. 18, 2006, the content of which is hereby incorporated by reference into this application.

### BACKGROUND OF THE INVENTION

1. Field of the Invention

The present invention relates to a gas bubble-generating agent that is in a liquid state at low temperatures and that evaporates and generates gas bubbles in response to temperature increase or physical stimulation, a method for producing the same, and a therapeutic method using such gas bubble-generating agent.

2. Background Art

If gas bubbles can be generated in vivo, blood vessels can be blocked. This is effective as a therapeutic means for diseases that can be treated by blocking nutrient vessels, such as malignant neoplasm and hysteromyoma. Such gas bubbles can also be used as a contrast medium for diagnostic imaging apparatuses such as ultrasonic diagnostic system or MRI diagnostic systems.

As a means for generating gas bubbles, foaming agents are extensively used at the industrial level. At the time of plastic production, etc., in particular, organic compounds that decompose at 100° C. or higher and generate nitrogen gas are generally used. Use of such common bubbling agents, however, is almost impossible in vivo, particularly in blood vessels. This is because exposure of a body to a high temperature of 100° C. or higher is very invasive and a chemical substance that generates a gas upon thermal decomposition has high toxicity. An example of a technique for generating gas bubbles in vivo is a method wherein a highly volatile substance is stabilized in a liquid state and convergent ultrasound beams are applied thereto to generate gas bubbles selectively at a target site, as disclosed in K. Kawabata et al., 2005, Jpn. J. Appl. Phys. 44: 4548-4552. Also, a technique involving the use of a substance that has been bubbled ex vivo prior to the administration or a substance that has been initially prepared in the form of gas bubbles as disclosed in U.S. Pat. No. 4,718,433 is also common.

When treating malignant neoplasm or hysteromyoma by blocking nutrient vessels with gas bubbles as mentioned above, it is crucial for gas bubbles to remain at the same site in a closely attached state. In the aforementioned method comprising administering a substance previously bubbled ex vivo, however, bubbles are separated from each other via diffusion and cannot maintain a closely attached state. Thus, it is difficult to produce a therapeutic effect by such method. Also, a method whereby generating gas bubbles selectively at a target site after the administration of a volatile liquid to the body is time consuming because of the necessity of shifting of the focal area of ultrasonic application in order to block extensive areas of nutrient vessels.

When a gas bubble-generating agent is used as a contrast medium, the former method is suitable as a means for selectively observing a limited region to which convergent ultrasound beams for gas bubble generation have been applied, and the latter method is suitable for observing the entire body, and particularly the entirety of blood vessels. These two methods, however, are not suitable for observing an area of a moderate size, such as the entirety of a given organ.

### SUMMARY OF THE INVENTION

When a volatile liquid that can be administered to a body or a gas bubble-generating agent that is administered in the form of gas bubbles is used in conventional techniques, gas bubbles are present selectively at sites to which physical stimulation, such as ultrasound beams, has been applied, or gas bubbles are present throughout the body. Thus, it is impossible to generate gas bubbles selectively at given regions in the vicinity of the site to which the gas bubble-generating agent has been administered. Accordingly, the effect of a gas bubble-generating agent of a conventional technique as a contrast medium or blocking agent is limited.

The present invention is intended to provide a gas bubble-generating agent that can generate gas bubbles within a short period of time after administration thereof to a body in a liquid state and gas bubble generation therefrom can be accelerated via physical stimulation such as ultrasonic application.

The present inventors considered that a gas bubble-generating agent that remains in a liquid state during storage at low temperatures, that evaporates gradually upon administration thereof to the body, and that generates gas bubbles within about 30 minutes would be suitable as a gas bubble-generating agent that can attain the above object, and they have conducted concentrated studies.

As a result, they discovered that a conjugate in which a volatile hardly water-soluble substance, an amphiphilic substance, and an amphiphilic substance comprising a water-soluble polymer chemically bound thereto weakly interact with each other has desirable properties. This has led to the completion of the present invention.

Specifically, the present invention includes the following.

(1) A method for producing a gas bubble-generating agent comprising the following steps of:

(a) preparing a mixed solution of an amphiphilic substance, an amphiphilic substance comprising a water-soluble polymer chemically bound thereto, a hardly water-soluble substance having a boiling point of lower than 60° C. at atmospheric pressure, and a physiologically acceptable isotonic solution;

(b) pressurizing the mixed solution; and

(c) centrifuging the mixed solution after the step of pressurization,

wherein a molar concentration of the amphiphilic substance in the mixed solution prepared in step (a) is 10 times or more higher than that of the amphiphilic substance comprising a water-soluble polymer chemically bound thereto.

(2) The method for producing a gas bubble-generating agent according to (1), wherein the amphiphilic substances are the same or different phospholipids and the polymer is polyalkylene oxide.

(3) The method for producing a gas bubble-generating agent according to (1), wherein the hardly water-soluble substance having a boiling point of lower than 60° C. at atmospheric pressure is perfluorocarbon.

(4) The method for producing a gas bubble-generating agent according to (1), wherein a fraction having a particle diameter of 200 nm or smaller is obtained after the step of centrifugation.

(5) An ultrasonic applicator comprising: a gas bubble-generating agent holding portion that holds a gas bubble-generating agent in which an amphiphilic substance, and a hardly water-soluble substance having a boiling point of lower than 60° C. at atmospheric pressure are bound to an amphiphilic substance comprising a water-soluble polymer chemically bound thereto; a catheter holding portion that holds a catheter that transports the gas bubble-generating agent from the gas bubble-generating agent holding portion to a given site of a subject's body; and an ultrasonic application portion that applies ultrasound beams to the given site.

(6) The ultrasonic applicator according to (5), wherein the ultrasonic application portion applies ultrasound beams at an intensity of 200 W/cm² or lower.

(7) The ultrasonic applicator according to (5), which further comprises: an ultrasonic receiver that receives ultrasound beams from the given site; an imaging portion that forms an ultrasonic image based on the information that the ultrasonic receiver has received; and a display portion that displays the ultrasonic image.

(8) A therapeutic method comprising transporting a gas bubble-generating agent in which an amphiphilic substance, and a hardly water-soluble substance having a boiling point of lower than 60° C. at atmospheric pressure are bound to an amphiphilic substance comprising a water-soluble polymer chemically bound thereto, to a given site of a subject's body via a catheter.

(9) The therapeutic method according to (8), which further comprises applying ultrasound beams to the given site.

(10) The therapeutic method according to (8), wherein the given site is a nutrient vessel headed toward a myoma or tumor.

(11) The therapeutic method according to (8), wherein the particle diameter of the gas bubble-generating agent is 200 nm or smaller.

(12) The therapeutic method according to (9), wherein the hardly water-soluble substance having a boiling point of lower than 60° C. at atmospheric pressure is perfluorocarbon and its phase shift is promoted by ultrasonic application.

(13) The therapeutic method according to (9), wherein the ultrasonic intensity is 200 W/cm² or lower.

(14) A blood vessel blocking agent comprising, as an active ingredient, the gas bubble-generating agent produced by the method according to any of (1) to (4).

### BRIEF DESCRIPTION OF THE DRAWINGS

FIG. 1 shows an example of the particle diameter distribution of the gas bubble-generating agent of the present invention.

FIG. 2 shows an example of the changes in percentage of perfluorocarbons that have disappeared and changes in scattering intensity with the elapse of time when the gas bubble-generating agent of the present invention is maintained at a body temperature (37° C.).

FIG. 3 shows an example of changes in scattering intensity with the elapse of time when the gas bubble-generating agent of the present invention is maintained at a body temperature (37° C.) and ultrasonic application is performed.

FIG. 4 shows an example of changes in scattering intensity with the elapse of time when the gas bubble-generating agent of the present invention is maintained at a body temperature (37° C.).

FIG. 5 shows an example of the structure of a catheter used in combination with the gas bubble-generating agent of the present invention.

FIG. 6 shows an example of the structure of an ultrasonic applicator used in combination with the gas bubble-generating agent of the present invention.

FIG. 7 shows an example of the structure of an ultrasonic applicator used in combination with the gas bubble-generating agent of the present invention.

### PREFERRED EMBODIMENTS OF THE INVENTION

An aspect of the present invention relates to a method for producing a gas bubble-generating agent comprising the steps of:

(a) preparing a mixed solution of an amphiphilic substance, an amphiphilic substance comprising a water-soluble polymer chemically bound thereto, a hardly water-soluble substance having a boiling point of lower than 60° C. at atmospheric pressure, and a physiologically acceptable isotonic solution;

(b) pressurizing the mixed solution; and

(c) centrifuging the mixed solution after the step of pressurization,

wherein a molar concentration of the amphiphilic substance in the mixed solution prepared in step (a) is 10 times or more higher than that of the amphiphilic substance comprising a water-soluble polymer chemically bound thereto.

The term “amphiphilic substance” refers to a substance that has both a hydrophilic group and a hydrophobic group, and an amphiphilic substance has affinity with a polar solvent and with a nonpolar solvent. An amphiphilic substance is not particularly limited, and a less toxic substance is preferable from the viewpoint of administration to living organisms. Examples of less toxic amphiphilic substances include phospholipids and glycolipids. Examples of stabilizers used in combination with amphiphilic substances to construct highly stable membrane structures include cholesterol and tocopherol. Examples of phospholipids include glycerophospholipids and sphingophospholipids. Specific examples include phosphatidyl choline, phosphatidylethanolamine, phosphatidylserine, phosphatidylglycerol, phosphatidic acid, phosphatidylinositol, cardiolipin, and sphingomyelin. Examples of glycolipids include glyceroglycolipids and sphingoglycolipids. Specific examples include neutral sphingoglycolipids such as galactocerebroside, glucocerebroside, and globoside, acidic sphingoglycolipids such as ganglioside, and mono- and digalactosyldiacylglycerol. An amphiphilic substance may be a naturally occurring or synthetic substance. An amphiphilic substance may be used alone or in combinations of two or more.

Examples of amphiphilic substances in an amphiphilic substance comprising a water-soluble polymer chemically bound thereto are as defined above. The aforementioned amphiphilic substance may be the same as or different from an amphiphilic substance in an amphiphilic substance comprising a water-soluble polymer chemically bound thereto. Glycerophospholipid is preferable, and phosphatidylethanolamine is more preferable. Specific examples of phosphatidylethanolamine include dioleoylphosphatidylethanolamine, dilinoleylphosphatidylethanolamine, dilinolenylphosphatidylethanolamine, dilinolenoyl phosphatidylethanolamine, diarachidoyl phosphatidylethanolamine, dimyristoyl phosphatidylethanolamine, dipalmitoyl phosphatidylethanolamine, and distearoyl phosphatidylethanolamine.

A water-soluble polymer in an amphiphilic substance comprising a water-soluble polymer chemically bound thereto is not particularly limited, provided that it is not highly toxic to living organisms. Examples thereof include polyalkylene oxide (e.g., a copolymer of polyethylene glycol, polypropylene glycol or ethylene glycol and propylene glycol copolymer) and its monoester or diester, polyvinyl alcohol, polyacrylamide, cellulose, dextran, gellan gum, polyvinyl alcohol, polyvinyl pyrrolidone, hydroxyethylcellulose, hydroxypropylcellulose, methylcellulose, carboxymethylcellulose, carboxy vinyl polymer, an alkyl acrylate/methacrylate copolymer, and a derivative of any of such polymers each comprising a functional group attached thereto. Use of a water-soluble polymer comprising a functional group attached thereto enables a physiologically active substance to bind to the water-soluble polymer via such functional group. In the present invention, a water-soluble polymer is preferably polyalkylene oxide, its monoester, or a derivative of either thereof comprising a functional group attached thereto. A water-soluble polymer is more preferably polyethylene glycol, its monoester, or a derivative of either thereof comprising a functional group attached thereto. Examples of a functional group include maleimide, amino, amide, carboxy, haloformyl, hydroxyl, cyano, nitro, and mercapto groups. In order to bind an amino-acid-containing molecule such as a peptide or antibody, an amide, maleimide, or mercapto group is preferable as a functional group.

The molecular weight of a water-soluble polymer portion in an amphiphilic substance comprising a water-soluble polymer chemically bound thereto is generally 500 to 20,000, and preferably 1,000 to 10,000. When a water-soluble polymer is polyethylene glycol, for example, the molecular weight thereof is generally 500 to 20,000, and preferably 1,000 to 10,000.

As a water-soluble polymer, a polymer comprising a physiologically active substance bound thereto via a functional group as mentioned above may be used. A physiologically active substance that can bind to a water-soluble polymer can be adequately selected in accordance with the purpose of use, and it is not particularly limited. A substance that exhibits selectivity to a certain molecule may be used. Examples thereof include: peptides, antibodies, and immunoreactive fragments thereof; anticancer agents such as adriamycin, daunomycin, pinorubin, methotrexate, mitomycin C, etoposide, cisplatin, and a derivative of any of such substances; various growth factors that can accelerate vital tissue organization and can block blood vessels, such as platelet-derived growth factors, epidermal growth factors, transforming growth factors α, insulin-like growth factors, insulin-like growth factor-binding proteins, hepatocellular growth factors, vascular endothelial growth factors, angiopoietin, neural growth factors, brain-derived neurotrophic factors, ciliary neurotrophic factors, transforming growth factors β, latent transforming growth factors β, activin, bone morphogenic proteins, fibroblast growth factors, tumor growth factors β, diploid fibroblast growth factors, heparin-binding epidermal growth factor-like growth factors, schwannoma-derived growth factors, amphiregulin, betacellulin, epiregulin, and lymphotoxin; and clot accelerators such as coagulants, for example, thrombin, fibrinogen, blood coagulation factors, hemocoagulase, oxidized cellulose, sodium alginate, aluminum chloride, and gelatin.

In an embodiment, an example of an amphiphilic substance comprising a water-soluble polymer chemically bound thereto is a substance represented by the following formula:

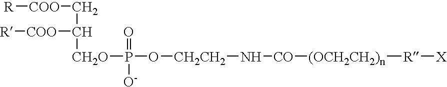

Wherein R and R′, which may be the same or different, each represent a linear or branched and saturated or unsaturated aliphatic group having 7 to 30 and preferably 10 to 20 carbon atoms, such as an alkyl, alkenyl, or alkynyl group; R″ represents a direct bond or a hydrocarbon group having a chain length of 1 to 15, which may contain a hetero atom selected from among oxygen, nitrogen, and sulfur atoms; n is an integer of 10 to 300, and preferably 20 to 100; and X represents an alkyl, alkenyl, alkoxy, or acyl group having 1 to 6 carbon atoms, or maleimide, amino, amide, carboxy, haloformyl, hydroxyl, cyano, nitro, or mercapto group.

A hardly water-soluble substance having a boiling point of lower than 60° C. at atmospheric pressure is not particularly limited, provided that such substance is not highly toxic to living organisms. Use of a hardly water-soluble substance having a boiling point of 29° C. to 58° C. at atmospheric pressure is preferable. As such hardly water-soluble substance, a substance the phase shift of which from a liquid phase to a gas phase is promoted via ultrasonic application is preferably used. Preferably, fluorocarbon and a hydrogenated derivative thereof are used, and perfluorocarbon and a hydrogenated derivative thereof are more preferably used. Perfluorocarbon having 3 to 10 carbon atoms and a hydrogenated derivative thereof are preferable. Specific examples include perfluoropropane, perfluorobutane, perfluoropentane, perfluorohexane, perfluoroheptane, perfluorooctane, perfluoromethylcyclohexane, perfluoromethylcyclopentane, perfluorodimethylcyclohexane, methylperfluorobutylether, ethylperfluorobutylether, perfluorodimethylcyclopentane, and a hydrogenated derivative of any of such substances. A hardly water-soluble substance may be used alone or in combinations of two or more.

The term “isotonic solution” refers to a solution in which a net migration of water is not observed at all when cells (or living organisms) are soaked therein. In general, a physiologically acceptable isotonic solution is used. A physiologically acceptable isotonic solution common in the art can be used. Examples thereof that can be used include physiological saline, phosphate buffer, and citrate buffer. The pH level of an isotonic solution is generally about 6 to 8.

In a mixed solution of an amphiphilic substance, an amphiphilic substance comprising a water-soluble polymer chemically bound thereto, a hardly water-soluble substance having a boiling point of lower than 60° C. at atmospheric pressure, and a physiologically acceptable isotonic solution, the molar concentration of the amphiphilic substance is 10 times or more higher, preferably 20 times or more higher, and more preferably 50 times or more higher than that of the amphiphilic substance comprising a water-soluble polymer chemically bound thereto. Such mixing ratio enables generation of gas bubble to an adequate degree from the viewpoint of blood vessel blockage.

At such mixing ratio, the amphiphilic substance comprising a water-soluble polymer chemically bound thereto can be mixed with the water-soluble substance or with the hardly water-soluble substance, and can function as a carrier that wraps the hardly water-soluble substance with a retentivity somewhat lower than that of a liposome or emulsion.

In the method for producing a gas bubble-generating agent of the present invention, pressurization to the mixed solution is carried out generally at the atmospheric pressure to 250 MPa, and preferably 50 to 200 MPa, generally for 10 seconds to 30 minutes, preferably 1 to 15 minutes, and more preferably 1 to 3 minutes, for example in an emulsification equipment commonly used in the art. After pressurization, the mixed solution is subjected to centrifugation. Centrifugation is generally carried out at 1,500 to 20,000 G for 10 seconds to 20 minutes.

In the method for producing a gas bubble-generating agent of the present invention, following the step of centrifugation, it is preferable to obtain a fraction having a particle diameter of 200 nm or smaller, preferably 150 nm or smaller, and more preferably 100 nm or smaller, for example by using a filter. Thus, the pore size of a filter to be used may be varied according to need. For example, a membrane filter having a pore size of 0.1 μm to 0.45 μm can be used. Filtration can be carried out several times.

The gas bubble-generating agent obtained by the method of the present invention is considered to have a structure in which an amphiphilic substance, and a hardly water-soluble substance having a boiling point of lower than 60° C. at atmospheric pressure are bound to an amphiphilic substance comprising a water-soluble polymer chemically bound thereto. By obtaining the fraction having the aforementioned particle diameter, a gas bubble-generating agent having a particle diameter of 200 nm or smaller, preferably 150 nm or smaller, and more preferably 100 nm or smaller, and an average particle diameter of 70 to 200 nm, and preferably 70 to 100 nm, can be obtained.

The gas bubble-generating agent obtained by the method of the present invention can spontaneously generate gas bubbles at around body temperature, i.e., between 35° C. and 40° C., and gas bubble generation is completed within about 100 minutes. Gas bubbles generated with the aid of the gas bubble-generating agent of the present invention can remain at the same site in a closely attached state without diffusion. When the gas bubble-generating agent of the present invention is administered to the body, accordingly, gas bubbles are generated gradually and the generated gas bubbles become closely attached to each other. Thus, blood vessels can be blocked selectively at a given region in the vicinity of the site to which the gas bubble-generating agent has been administered. Gas bubbles would not diffuse throughout the body, and they would not reach undesirable sites. The gas bubble-generating agent of the present invention is advantageous, since gas bubbles are spontaneously generated at body temperature and, therefore, continuous ultrasonic application is not required.

The gas bubble-generating agent of the present invention spontaneously generates gas bubbles in vivo, and gas bubble generation can further be accelerated by ultrasonic application. With the use of a conventional gas bubble-generating agent that generates gas bubbles with the aid of ultrasound beams, gas bubbles were generated only while ultrasound beams had been applied, and gas bubbles disappeared upon termination of ultrasonic application. The gas bubble-generating agent of the present invention, however, maintains the generated gas bubbles after the termination of ultrasonic application, once the gas bubble generation is accelerated via ultrasonic application. Accordingly, gas bubbles can be generated over given regions such as a target disease site or an entire organ and can block blood vessels therein as well as a limited region to which ultrasound beams have been applied.

The present invention relates to a therapeutic method or a method for blocking blood vessels comprising transporting a gas bubble-generating agent produced by the method of the present invention to a given site of a subject's body via a catheter. In other words, the present invention relates to a therapeutic method or a method for blocking blood vessels comprising transporting a gas bubble-generating agent in which an amphiphilic substance, and a hardly water-soluble substance having a boiling point of lower than 60° C. at atmospheric pressure are bound to an amphiphilic substance comprising a water-soluble polymer chemically bound thereto, to a given site of a subject's body via a catheter.

The gas bubble-generating agent and the method for producing the same are as defined above.

The therapeutic method of the present invention can treat a disease that can be treated by blocking blood vessels, such as a disease associated with neovascularization. Examples thereof include tumor, myoma, aneurysm, intraocular neovascular disease, rheumatoid arthritis, angioma, Basedow's disease, age-related macular degeneration, diabetic retinopathy, retinopathy of prematurity, occlusion of retinal vein, polypoid choroid vasculopathy, diabetic macular edema, psoriasis vulgaris, and atherosclerosis. The gas bubble-generating agent is transported to a blood vessel headed toward a disease site, and gas bubbles are generated to block the blood vessel and to inhibit the blood flow toward the disease site. Thus, the disease can be treated. When treating tumor or myoma, for example, the gas bubble-generating agent is transported to the nutrient vessels headed toward the target tumor or disease site to generate gas bubbles and to inhibit the supply of nutrients to a tumor or myoma. Thus, therapeutic effects can be attained. The therapeutic method of the present invention is preferably employed to treat tumors, and particularly solid tumors, such as lung cancer, brain tumor, epipharynx carcinoma, lingual cancer, esophageal cancer, gastric cancer, pancreatic cancer, hepatic cancer, rectal cancer, colon cancer, uterine cancer, ovarian cancer, testicular carcinoma, or osteosarcoma, and myoma, and particularly hysteromyoma.

Preferably, the therapeutic method of the present invention further comprises applying ultrasound beams to the aforementioned given site, i.e., a disease site or a region in the vicinity thereof. In this method, ultrasound beams with an intensity of generally 5 W/cm² to 200 W/cm² and preferably 10 W/cm² to 100 W/cm² and a frequency of generally 0.5 MHz to 10 MHz are applied. The ultrasonic application can accelerate gas bubble generation from the gas bubble-generating agent.

According to the therapeutic method of the present invention, treatment can be carried out by visualizing the generated gas bubbles in real time using an ultrasonic diagnostic equipment to evaluate the effects of blood vessel blocking.

The targets of the therapeutic method of the present invention include, but are not particularly limited to, mammalian animals such as humans, livestock animals such as bovines and horses, pet animals such as dogs and cats, and test animals such as mice, rats, and hamsters.

The present invention also relates to a blood vessel blocking agent as a pharmaceutical composition comprising, as an active ingredient, the gas bubble-generating agent produced by the method of the present invention. The aforementioned diseases can be treated by administering the blood vessel blocking agent of the present invention to a given site via a catheter. The blood vessel blocking agent of the present invention can comprise a wide variety of known additives within the scope of the present invention. Examples of such additives include carriers, excipients, antiseptic agents, stabilizers, binders, antioxidants, swelling agents, isotonizing agents, solubilizers, preservatives, buffers, and diluents.

The present invention also relates to an ultrasonic applicator used in the above therapeutic method. The ultrasonic applicator comprises: a gas bubble-generating agent holding portion that holds a gas bubble-generating agent produced by the method of the present invention, i.e., a gas bubble-generating agent in which an amphiphilic substance, and a hardly water-soluble substance having a boiling point of lower than 60° C. at atmospheric pressure are bound to an amphiphilic substance comprising a water-soluble polymer chemically bound thereto; a catheter holding portion that holds a catheter that transports the gas bubble-generating agent from the gas bubble-generating agent holding portion to a given site of a subject's body; and an ultrasonic application portion that applies ultrasound beams to the given site.

The ultrasonic application portion applies ultrasound beams with an intensity of generally 5 W/cm² to 200 W/cm² and preferably 10 W/cm² to 100 W/cm² and a frequency of generally 0.5 MHz to 10 MHz.

By using the ultrasonic applicator of the present invention in combination with ultrasonic diagnostic equipment, the generated gas bubbles can be visualized in real time, the effects of blood vessel blocking can be evaluated, and more effective treatment can be provided by regulating gas bubble generation. Specifically, it is preferable that the ultrasonic applicator of the present invention further comprise an ultrasonic diagnostic equipment comprising: an ultrasonic receiver that receives ultrasound beams from a given site; an imaging portion that forms an ultrasonic image based on the information that the ultrasonic receiver received; and a display portion that displays the ultrasonic image.

The catheter generally comprises a pressurization means whereby pressurizing the gas bubble-generating agent and discharging the same from the end of the catheter. Alternatively, the catheter may comprise a heating means for accelerating gas bubble generation by the gas bubble-generating agent. The catheter body or the catheter end may be provided with a biocompatible and lubricating coating layer. The coating layer enables prevention of thrombus formation on the catheter surface, prevention of damage to an intimal layer of a blood vessel by the catheter, and smooth intravascular migration by reducing friction with the inner walls of a blood vessel.

According to the present invention, gas bubbles can be generated at a target site in vivo in amounts required. Thus, a safe therapeutic technique can be provided.

Hereafter, the test examples and examples of the present invention are described in detail, although the technical scope of the present invention is not limited thereto.

### EXAMPLES

### Example 1

### Preparation of Gas Bubble-Generating Agent

The ingredients shown below were maintained at 4° C. and mixed, and the resulting mixture was homogenized with an Ultra Turrax T25 (Janke & Kunkel, Staufen, Germany) at 9,500 rpm for 1 minute while slowly adding 20 ml of phosphate buffer (pH: 7.4).

TABLE 1

| | |
|---|---|
| Glycerol | 2.0 g |
| α-Tocopherol | 0.02 g |
| Cholesterol | 0.1 g |
| Phosphatidyl choline | 1.0 g |
| Perfluoropentane | 0.1 g |
| 2H,3H-perfluoropentane | 0.1 g |
| MPEG-2000-DSPE | 0.01 g |

The chemical structure of MPEG-2000-DSPE is shown below.

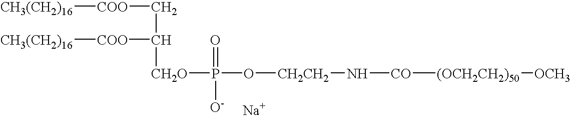

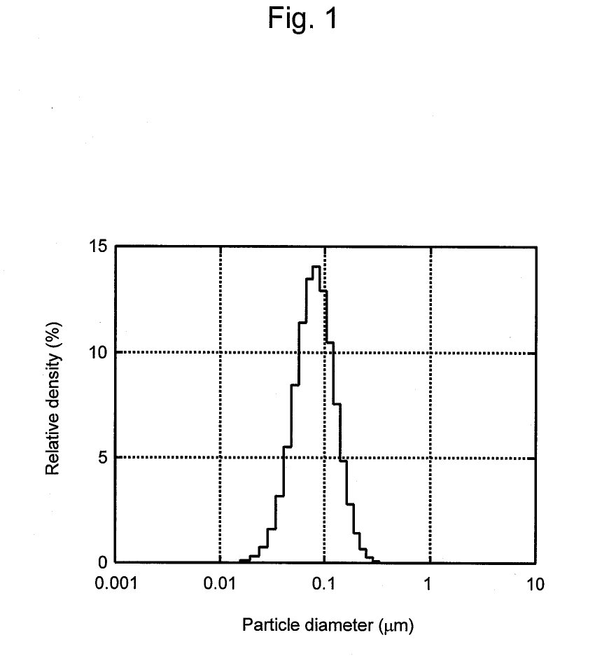

The resulting emulsion was subjected to high-pressure emulsification in an Emulsiflex-C5 (Avestin, Ottawa, Canada) at 20 MPa for 10 minutes, the resultant was centrifuged at 10,000 G for 15 minutes, the residue was removed, and filtration was carried out using a 0.1-μm membrane filter to obtain a substantially transparent microparticle dispersion. The dispersion was refrigerated until the time of use, and the temperature was raised to room temperature immediately before use. The particle diameter distribution of the obtained microparticles was assayed using an LB-550 (Horiba Seisakusho, Tokyo, Japan). The results are shown in FIG. 1. As is apparent from the figure, microparticles having an average particle diameter of 0.070 μm were obtained.

Hereafter, the results of the test concerning the effects of the gas bubble-generating agent prepared in the above described manner are described.

### Test Example 1

### Test Concerning the Time Required for Gas Bubble Generation at a Body Temperature (37° C.)

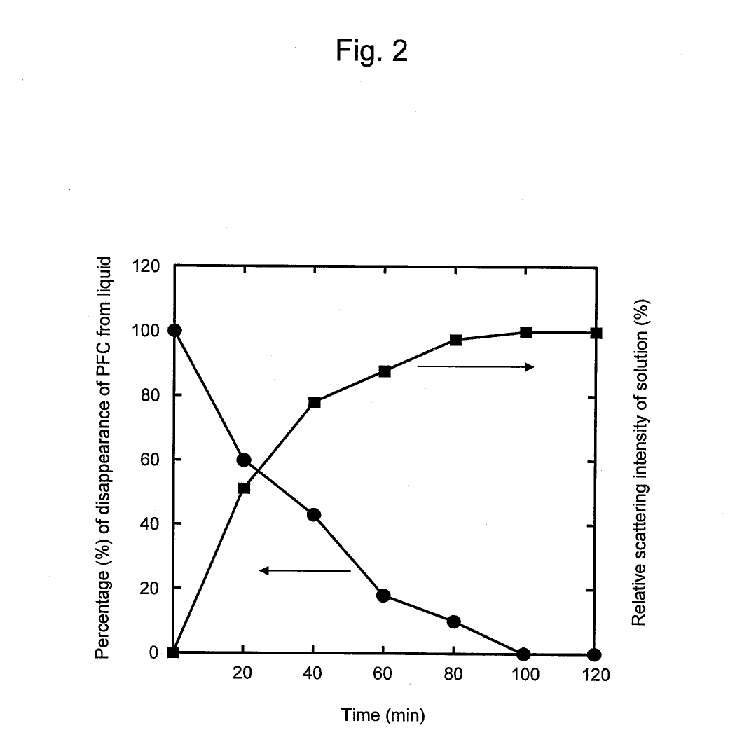

FIG. 2 shows the results of a test concerning the time required for the gas bubble-generating agent to generate gas bubbles. The test results were obtained by raising the temperature of the gas bubble-generating agent prepared in Example 1 from a refrigeration state of 4° C. to room temperature, placing the gas bubble-generating agent in a 1-cm cell for fluorescent assay, immobilizing the cell in an incubator set at 37° C., and assaying the time required for gas bubble generation using changes in the scattering intensity of the solution and the percentage of perfluorocarbons that had disappeared from the liquid phase as indicators.

Changes in the scattering intensity of the solution were assayed by applying a semiconductor laser at 655 nm (350 μW) to the cell and determining changes in the intensity of the laser which entered into a photoreceiver set perpendicular to the laser axis. The percentage of perfluorocarbons that had disappeared from the liquid phase was assayed by removing 10 μl of the liquid phase of the sample in the 1-cm cell and assaying the density of perfluorocarbons using a gas chromatography apparatus. As is apparent from FIG. 2, the scattering intensity was increased with the elapse of time and perfluorocarbons disappeared from the liquid phase. In general, scattering takes place at the interface. This indicates that the increased scattering intensity results in increased interfaces. Also, minute gas bubbles were visually observed. As the scattering intensity was increased, more perfluorocarbons disappeared from the liquid phase.

These results demonstrate that the gas bubble-generating agent prepared in the present example generates gas bubbles at body temperature. Based on the results of inspection of the scattering intensity and disappearance of perfluorocarbons, the time required for the gas bubble-generating agent of the present example to complete gas bubble generation was found to be substantially 100 minutes.

### Test Example 2

### Test Concerning the Effects of Ultrasonic Application on Gas Bubble Generation

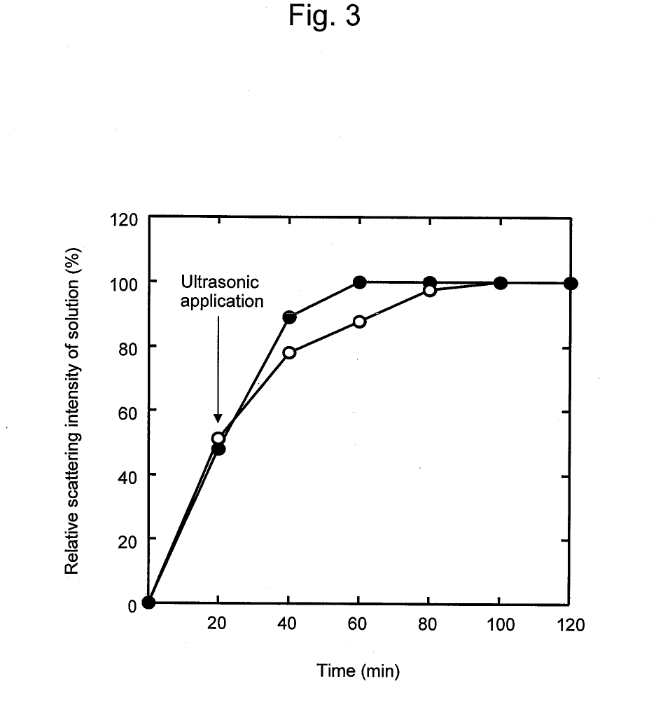

FIG. 3 shows the results of a test concerning the effects of ultrasonic application on gas bubble generation from the gas bubble-generating agent of the present example. The test results were obtained by raising the temperature of the gas bubble-generating agent prepared in Example 1 from a refrigeration state of 4° C. to room temperature, placing the gas bubble-generating agent in a 1-cm cell for fluorescent assay, immobilizing the cell in an incubator set at 37° C., assaying the time required for gas bubble generation using changes in the scattering intensity of the solution as an indicator, and applying ultrasonic beams 20 minutes thereafter. Changes in the scattering intensity of the solution were assayed by applying a semiconductor laser at 655 nm (350 μW) to the cell and determining changes in the intensity of the laser which entered into a photoreceiver set perpendicular to the laser axis, as in the case of Test Example 2. The outlined circle represents a case in which ultrasonic application is not performed, and the black circle represents a case in which ultrasonic application is performed at a frequency of 2 MHz and an intensity of 10 W/cm² for 1 minute. In both cases, scattering was increased with the elapse of time, which indicates that gas bubble generation advances as described in Test Example 1. As is apparent from FIG. 3, gas bubble generation is accelerated by ultrasonic application, the time required for the completion of gas bubble generation (i.e., the time point at which the scattering intensity would no longer be increased) is about 100 minutes without ultrasonic application, and such time is about 60 minutes with ultrasonic application. This indicates that gas bubble generation is completed in the latter case within about a half of the time needed for the former case. Substantially the same test results were obtained when ultrasonic beams were applied while changing the frequency in the range between 0.5 MHz and 10 MHz and the intensity in the range between 5 W/cm² and 200 W/cm² for 10 ms or longer.

### Test Example 3

### Test Concerning Effects of Perfluorocarbon Type

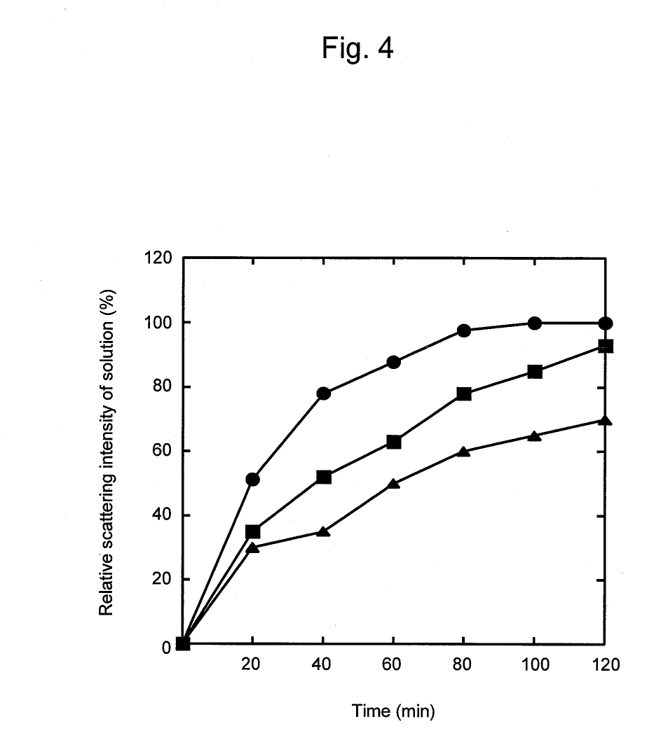

FIG. 4 shows the results of the test concerning the effects of perfluorocarbon type on gas bubble generation by the gas bubble-generating agent of the present example. The test results were obtained by raising the temperature of the gas bubble-generating agent prepared in Example 1 and that of a gas bubble-generating agent prepared in the same manner except for the use of perfluorohexane or perfluoroheptane instead of perfluoropentane from a refrigeration state of 4° C. to room temperature, placing the gas bubble-generating agents in 1-cm cells for fluorescent assay, immobilizing the cells in an incubator set at 37° C., and assaying the time required for gas bubble generation using changes in the scattering intensity of the solutions as an indicator. Changes in the scattering intensity of the solutions were assayed by applying a semiconductor laser at 655 nm (350 μW) to the cells and determining changes in the intensity of the laser which entered into a photoreceiver set perpendicular to the laser axis, as in the cases of Test Examples 2 and 3.

A circle, a square, and a triangle represent a case in which perfluoropentane, perfluorohexane, or perfluoroheptane was used, respectively. In all cases, scattering was increased with the elapse of time, which indicates that gas bubble generation advances even when perfluorocarbon type was altered. The degree of gas bubble generation varies depending on a type of perfluorocarbon used. Evaporation was least likely to occur with perfluoropentane followed by perfluorohexane and then perfluoroheptane. 100% of evaporation was not achieved except when perfluoropentane was used. Thus, these three cases were compared in terms of the time required for 50% evaporation. As a result, such time was found to be 20, 40, and 60 minutes for perfluoropentane, perfluorohexane, and perfluoroheptane, respectively.

As is apparent from the test examples, the gas bubble-generating agent of the present invention can generate gas bubbles at body temperature.

### Example 2

### Preparation of Gas Bubble-Generating Agent Using a Polymer having a Functional Group

The ingredients shown below were maintained at 4° C. and mixed, and the resulting mixture was homogenized with an Ultra Turrax T25 (Janke & Kunkel, Staufen, Germany) at 9,500 rpm for 1 minute while slowly adding 20 ml of phosphate buffer (pH: 7.4).

TABLE 2

| | |
|---|---|
| Glycerol | 2.0 g |
| α-Tocopherol | 0.02 g |
| Cholesterol | 0.1 g |
| Phosphatidyl choline | 1.0 g |
| Perfluoropentane | 0.1 g |
| 2H,3H-perfluoropentane | 0.1 g |
| MPEG-2000-DSPE | 0.007 g |
| MPEG-2000-DSPE-MA | 0.003 g |

The chemical structure of MPEG-2000-DSPE-MA is shown below.

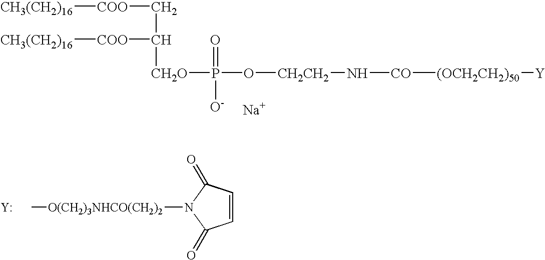

The resulting emulsion was subjected to high-pressure emulsification in an Emulsiflex-C5 (Avestin, Ottawa, Canada) at 20 MPa for 10 minutes, the resultant was centrifuged at 10,000 G for 15 minutes, the residue was removed, and filtration was carried out using a 0.1-μm membrane filter to obtain a substantially transparent microparticle dispersion. The dispersion was refrigerated until the time of use, and the temperature was raised to room temperature immediately before use. The particle diameter distribution of the obtained microparticles was assayed using an LB-550 (Horiba Seisakusho, Tokyo, Japan), and microparticles having an average particle diameter of 0.070 μm were found to be obtained.

### Example 3

### Ultrasonic Applicator

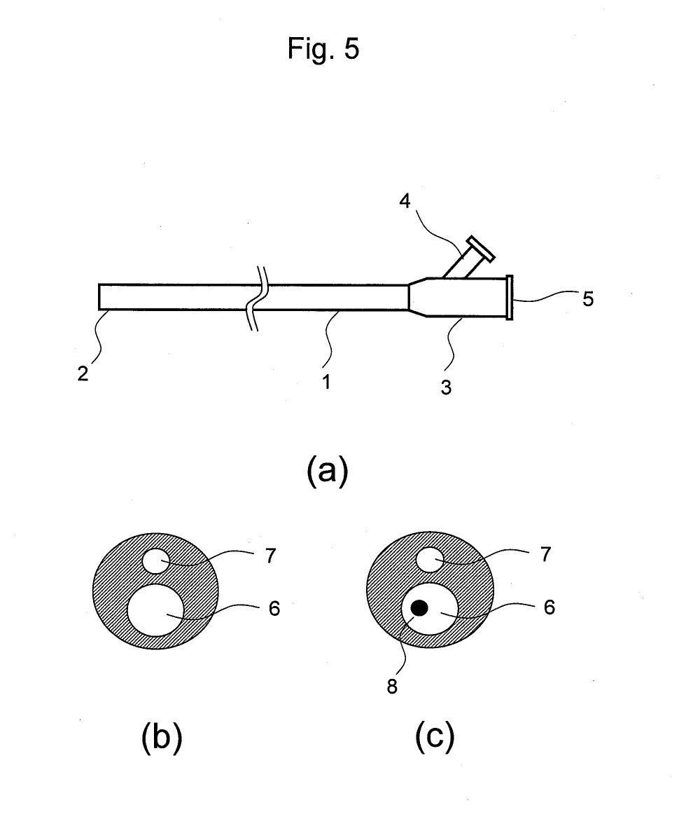

An example of an ultrasonic applicator used in combination with the gas bubble-generating agent of the present invention is described with reference to FIGS. 5, 6, and 7. FIG. 5 shows the structure of a catheter used for administering a gas bubble-generating agent to the affected area. FIG. 5(a) shows an appearance of a catheter, which comprises a catheter body 1, a catheter end 2, and a catheter base 3. The catheter base 3 comprises a gas bubble-generating agent-introduction port 4 and a guidewire port 5. At the time of use, a reservoir containing a gas bubble-generating agent is connected to the gas bubble-generating agent-introduction port 4, and the gas bubble-generating agent is pressurized using a pressurization means and discharged from the end 2. FIG. 5(b) shows a cross-section of the catheter body 1. As shown in this figure, the catheter body 1 comprises: a channel for a gas bubble-generating agent, i.e., a channel 6 that introduces the gas bubble-generating agent from the introduction port 4 of the catheter base 3 and discharges the same from the catheter end 2; and a guide channel, i.e., a channels 7 that allows insertion of a guidewire or observation scope. As shown in FIG. 5(c), the channel 5 can comprise a heating means 8 for accelerating gas bubble generation. The surface of the catheter body 1 and that of the catheter end 2 are provided with biocompatible and lubricating coating layers that are not shown. The coating layer enables prevention of thrombus formation on the catheter surface, prevention of a damage to an intimal layer of a blood vessel by the catheter, and smooth intravascular migration by reducing friction with the inner walls of a blood vessel.

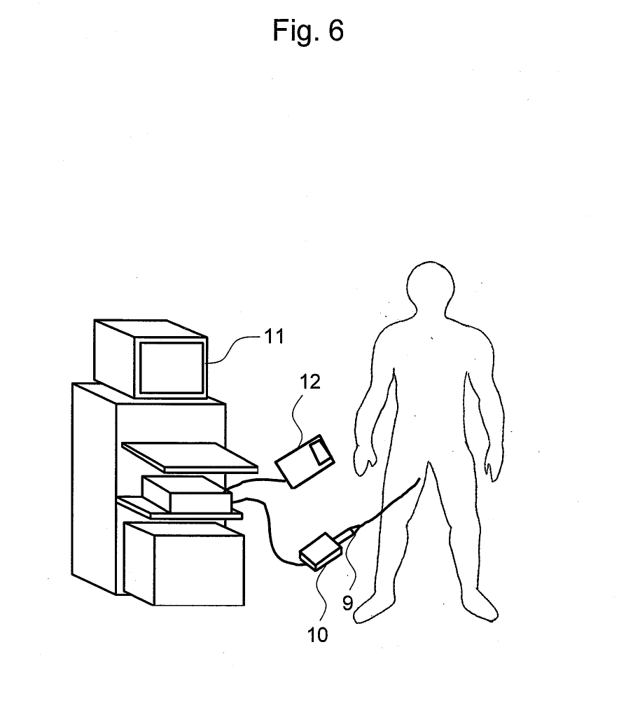

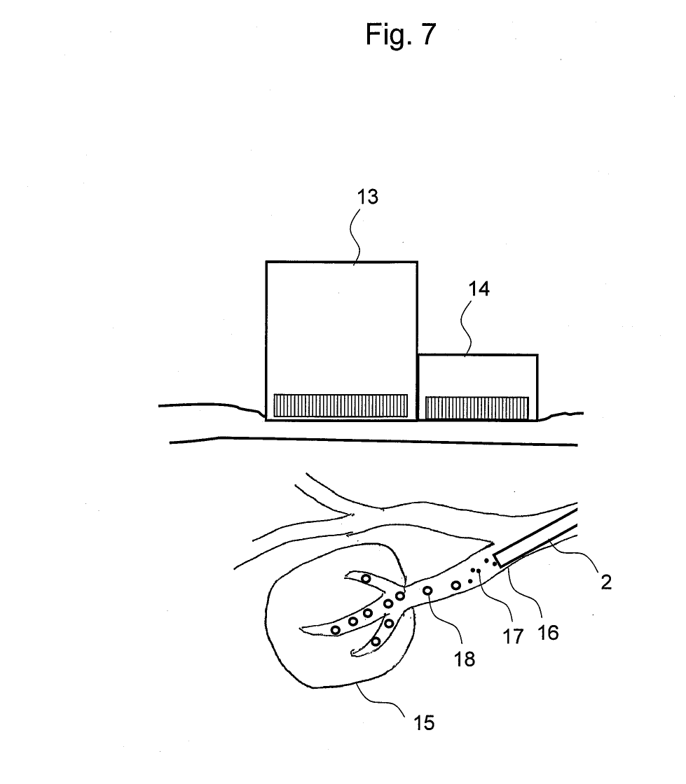

FIG. 6 shows an ultrasonic applicator used in combination with the catheter shown in FIG. 5. This ultrasonic applicator comprises a catheter 9, a gas bubble-generating agent storage/introduction control portion 10, an ultrasonic applicator 11, and an ultrasonic probe 12. More specifically, the ultrasonic probe 12 comprises an ultrasound transmission probe 13 for diagnosis and an ultrasound transmission probe 14 for gas bubble generation as shown in FIG. 7. At the time of actual use, a gas bubble-generating agent 17 is discharged through the gas bubble-generating agent storage/introduction control portion 10 from the catheter end 2 that is adequately provided relative to a target organ 15 and a target blood vessel 16. Gas bubbles 18 are generated by the heating means 8 and ultrasonic beams with frequencies of 0.5 MHz to 10 MHz and intensities of 5 W/cm² or higher applied from the ultrasound transmission probe 14 for gas bubble generation. The degree of gas bubble generation is monitored in real time with the use of the ultrasound transmission probe 13 for diagnosis, the heating means 8 is stopped when gas bubbles are adequately generated, and ultrasonic beams applied from the ultrasound transmission probe 14 for gas bubble generation are then stopped.

What is claimed is:

1. A method for producing a gas bubble-generating agent comprising the following steps of:
(a) preparing a mixed solution of an amphiphilic substance, an amphiphilic substance comprising a water-soluble polymer chemically bound thereto, a hardly water-soluble substance having a boiling point of lower than 60° C. at atmospheric pressure, and a physiologically acceptable isotonic solution;
(b) pressurizing the mixed solution; and
(c) centrifuging the mixed solution after the step of pressurization,
wherein a molar concentration of the amphiphilic substance in the mixed solution prepared in step (a) is 10 times or more higher than that of the amphiphilic substance comprising a water-soluble polymer chemically bound thereto.

2. The method for producing a gas bubble-generating agent according to claim 1, wherein the amphiphilic substances are the same or different phospholipids and the polymer is polyalkylene oxide.

3. The method for producing a gas bubble-generating agent according to claim 1, wherein the hardly water-soluble substance having a boiling point of lower than 60° C. at atmospheric pressure is perfluorocarbon.

4. The method for producing a gas bubble-generating agent according to claim 1, wherein a fraction having a particle diameter of 200 nm or smaller is obtained after the step of centrifugation.

5. An ultrasonic applicator comprising:
a gas bubble-generating agent holding portion that holds a gas bubble-generating agent in which an amphiphilic substance, and a hardly water-soluble substance having a boiling point of lower than 60° C. at atmospheric pressure are bound to an amphiphilic substance comprising a water-soluble polymer chemically bound thereto;
a catheter holding portion that holds a catheter that transports the gas bubble-generating agent from the gas bubble-generating agent holding portion to a given site of a subject's body; and
an ultrasonic application portion that applies ultrasound beams to the given site.

6. The ultrasonic applicator according to claim 5, wherein the ultrasonic application portion applies ultrasound beams at an intensity of 200 W/cm² or lower.

7. The ultrasonic applicator according to claim 5, which further comprises: an ultrasonic receiver that receives ultrasound beams from the given site; an imaging portion that forms an ultrasonic image based on the information that the ultrasonic receiver has received; and a display portion that displays the ultrasonic image.

8. A therapeutic method comprising transporting a gas bubble-generating agent in which an amphiphilic substance, and a hardly water-soluble substance having a boiling point of lower than 60° C. at atmospheric pressure are bound to an amphiphilic substance comprising a water-soluble polymer chemically bound thereto, to a given site of a subject's body via a catheter.

9. The therapeutic method according to claim 8, which further comprises applying ultrasound beams to the given site.

10. The therapeutic method according to claim 8, wherein the given site is a nutrient vessel headed toward a myoma or tumor.

11. The therapeutic method according to claim 8, wherein the particle diameter of the gas bubble-generating agent is 200 nm or smaller.

12. The therapeutic method according to claim 9, wherein the hardly water-soluble substance having a boiling point of lower than 60° C. at atmospheric pressure is perfluorocarbon and its phase shift is promoted by ultrasonic application.

13. The therapeutic method according to claim 9, wherein the ultrasonic intensity is 200 W/cm² or lower.

14. A blood vessel blocking agent comprising, as an active ingredient, the gas bubble-generating agent produced by the method according to claim 1.
